(19) United States
(12) Patent Application Publication
(10) Pub. No.: US 2025/0298020 A1
(43) Pub. Date: Sep. 25, 2025

(54) BIOMARKERS FOR MYCOBACTERIUM BOVIS

(71) Applicant: ABERYSTWYTH UNIVERSITY, Aberystwyth (GB)

(72) Inventors: Richard David PIZZEY, Aberystwyth (GB); Luis Alejandro Jose MUR, Aberystwyth (GB); David John ROOKE, Aberystwyth (GB)

(21) Appl. No.: 18/862,856

(22) PCT Filed: May 5, 2023
(86) PCT No.: PCT/GB2023/051188

§ 371 Date: Nov. 4, 2024

(30) Foreign Application Priority Data
May 5, 2022 (GB) 2206581.7

(51) Int. Cl. G01N 33/569; G01N 33/53; G01N 33/543; G01N 33/92

(52) U.S. Cl. CPC G01N 33/5695; G01N 33/5308; G01N 33/54386; G01N 33/92; G01N 2470/04

(57) ABSTRACT

The present invention relates to a method for determining the presence of Mycobacterium bovis in a subject. The method comprises: (i) determining the level of one or more biomarkers in a sample from the subject; and (ii) comparing the level of said one or more biomarkers with the level of said one or more metabolites in a control sample to determine whether Mycobacterium bovis is present in the subject. The biomarkers are selected from: dihydro-leukotriene B4, 3-hydroxyisovaleric acid, sphingomyelin [SM(d18:2(4E,14Z)/24:0)], sphingomyelin [SM(d18:1/24:1(15Z))], 18:3 cholesteryl ester, an unknown compound 389.21933 m/z (under negative ionisation), an unknown compound 886.71063 m/z (under negative ionisation), citric acid, MG(0:0/18:3(6Z,9Z,12Z)/0:0), pyrocatechol, N-(docosanoyl)-heptadecasphing-4-enine-1-phosphocholine and an unknown compound 359.22107 m/z (under negative ionisation).

### FIELD OF THE INVENTION

The present invention relates to biomarkers for detecting Mycobacterium bovis, the causative agent of bovine tuberculosis, which is a member of the Mycobacterium tuberculosis complex. In particular, the present invention relates to a method for determining the presence of Mycobacterium bovis in a subject. The present invention further relates to a device for detecting Mycobacterium bovis in a subject and a kit for detecting Mycobacterium bovis in a subject.

### BACKGROUND TO THE INVENTION

Mycobacterium bovis is a slow-growing aerobic bacterium which is the causative agent of bovine tuberculosis. Bovine tuberculosis is a chronic zoonotic disease of cattle that if left to run its course can cause severe pathology, particularly in the lungs, and compromise animal welfare, along with posing a risk to human health. In cattle, testing for bovine tuberculosis has been a key aspect of the control and eradication efforts in the UK over the past 70 years. The outcomes are used in the identification and culling of infected animals and, on a wider scale, provide evidence to understand bovine tuberculosis prevalence and inform risk regarding animal trading.

Infection is mainly through inhalation or ingestion of the bacteria and contaminated food and water can also be a source of infection. Cattle infected with M. bovis can spread the bacteria to other animals via the respiratory route (i.e. by inhalation of aerosol from an infected subject), via infected milk, via the placenta (i.e. in utero) or via environmental contamination.

The progression of bovine tuberculosis is often slow and it can take months or years to reach the stage when clinical symptoms become apparent. Therefore, an infected animal can shed bacteria before diagnosis, thus resulting in spread of the disease.

Accurate identification of infected animals, while fundamental, has proved challenging. The mainstay of diagnosis is, as in human medicine, based upon cell mediated immune response to M. bovis through tuberculin skin testing. In the UK, this is in the form of the single intradermal comparative cervical tuberculin test (SICCT) along with ancillary use of y-interferon αssays, an in vitro blood-based test of cell mediated immunity. More recently, antigen, antibody and bacteriophage-PCR diagnostics have been recognised as promising tools in the diagnosis of M. bovis infection, such as the Quantiferon gold test which uses three tuberculosis specific antigens to diagnose infection with M. bovis.

There are several criteria to consider when applying diagnostic tests including sensitivity and specificity, along with cost, turnaround times and ease of use. Based upon these criteria no current diagnostic test is truly exceptional and all involve compromise of either sensitivity, specificity, cost and practicality. In addition, none of the currently approved and used tests are able to “differentiate between infected and vaccinated animals” (DIVA). Novel and alternative diagnostics are necessary as recurrent herd breakdowns are still a major issue in parts of the UK and may result from failure to identify all infected cattle using the current diagnostic tools.

Mycobacterium bovis can also infect and cause diseases in many other mammals including humans, deer, pigs, goats and badgers, for example. In humans, M. bovis causes disease that can affect the lungs, lymph nodes and other parts of the body. Humans are most commonly infected with M. bovis by eating or drinking contaminated, unpasteurized dairy products. Some of these M. bovis strains have multi antibiotic drug resistance and therefore, this poses a considerable threat to the human population. Such resistant strains severely restrict the potential for treatment and vaccination and so diagnostic tools that can accurately determine potential bovine tuberculosis sources are crucial.

It is an object of the present invention to obviate or mitigate one or more of the abovementioned problems.

### SUMMARY OF THE INVENTION

The present invention relates to a method for determining the presence of Mycobacterium bovis in a subject and is based, in part, on studies by the inventors in which they have shown that certain metabolites are present at significantly different levels in calves infected with Mycobacterium bovis as compared to control calves and are therefore suitable as biomarkers for bovine tuberculosis.

In a first aspect of the present invention there is provided a method for determining the presence of Mycobacterium bovis in a subject, the method comprising the steps of:
(i) determining the level of one or more biomarkers in a sample from the subject;
(ii) comparing the level of said one or more biomarkers with the level of said one or more biomarkers in a control sample to determine whether Mycobacterium bovis is present in the subject;
   wherein the one or more biomarkers are selected from: dihydro-leukotriene B4, 3-hydroxyisovaleric acid, sphingomyelin [SM(d18:2(4E,14Z)/24:0)], sphingomyelin [SM(d18:1/24:1(15Z))], 18:3 cholesteryl ester, an unknown compound 389.21933 m/z (under negative ionisation), an unknown compound 886.71063 m/z (under negative ionisation), citric acid, MG(0:0/18:3(6Z,9Z,12Z)/0:0), pyrocatechol, N-(docosanoyl)-heptadecasphing-4-enine-1-phosphocholine and an unknown compound 359.22107 m/z (under negative ionisation).

As discussed in more detail below, the present inventors have found that the level of the abovementioned biomarkers is significantly altered in subjects in which Mycobacterium bovis is present. As shown below, the inventors have found that these biomarkers have individual diagnostic accuracies (area under the curve; AUC) of greater than 0.69. The inventors have therefore found that by comparing the level of the abovementioned biomarkers in a sample from a subject with the level in a control sample, the presence of Mycobacterium bovis in a subject can be determined with high sensitivity and specificity. These metabolite biomarkers are therefore useful for determining whether a subject has bovine tuberculosis, particularly subclinical bovine tuberculosis. By quickly and accurately identifying animals affected by bovine tuberculosis at an early stage of infection, and before clinical symptoms become apparent, spread of the disease can be reduced and/or prevented.

The levels of the metabolite biomarkers of the invention have been shown by the inventors to be consistently and significantly different between calves infected with Mycobacterium bovis as compared to control calves. These biomarkers can therefore be used to determine the presence of Mycobacterium bovis in a subject with extremely high sensitivity and specificity.

The method of the present invention comprises determining the level of one or more biomarkers in a sample from the subject. Preferably, the method of the present invention comprises determining the level of two, three, four, five, six, seven, eight, nine, ten, eleven or twelve (or more) biomarkers in a sample from the subject. By determining the level of multiple biomarkers in the sample and comparing multiple biomarkers with the level in a control sample, the sensitivity and specificity of the determination (of whether a subject has Mycobacterium bovis) may be improved. The level of one or more biomarkers in the sample will be determined. Together these biomarker levels may be integrated to form a “fingerprint” for the sample, thereby increasing the specificity of the method.

In embodiments of the present invention, the one or more biomarkers are selected from: dihydro-leukotriene B4, 3-hydroxyisovaleric acid, sphingomyelin [SM(d18:2(4E,14Z)/24:0)], sphingomyelin [SM(d18:1/24:1(15Z))], 18:3 cholesteryl ester, an unknown compound 389.21933 m/z (under negative ionisation), an unknown compound 886.71063 m/z (under negative ionisation), citric acid and MG(0:0/18:3(6Z,9Z,12Z)/0:0.

The present invention therefore provides a method for determining the presence of Mycobacterium bovis in a subject, the method comprising the steps of:
(i) determining the level of one or more biomarkers in a sample from the subject;
(ii) comparing the level of said one or more biomarkers with the level of said one or more biomarkers in a control sample to determine whether Mycobacterium bovis is present in the subject;
   wherein the one or more biomarkers are selected from: dihydro-leukotriene B4, 3-hydroxyisovaleric acid, sphingomyelin [SM(d18:2(4E,14Z)/24:0)], sphingomyelin [SM(d18:1/24:1(15Z))], 18:3 cholesteryl ester, an unknown compound 389.21933 m/z (under negative ionisation), an unknown compound 886.71063 m/z (under negative ionisation), citric acid and MG(0:0/18:3(6Z,9Z,12Z)/0:0.

In embodiments of the invention, the method may comprise determining the level of two, three, four, five, six, seven, eight or nine of the following biomarkers: dihydro-leukotriene B4, 3-hydroxyisovaleric acid, sphingomyelin [SM(d18:2(4E,14Z)/24:0)], sphingomyelin [SM(d18:1/24:1(15Z))], 18:3 cholesteryl ester, an unknown compound 389.21933 m/z (under negative ionisation), an unknown compound 886.71063 m/z (under negative ionisation), citric acid and MG(0:0/18:3(6Z,9Z,12Z)/0:0.

In embodiments in which the method involves determining the level of nine biomarkers in a sample from the subject, the biomarkers may consist of dihydro-leukotriene B4, 3-hydroxyisovaleric acid, sphingomyelin [SM(d18:2(4E,14Z)/24:0)], sphingomyelin [SM(d18:1/24:1(15Z))], 18:3 cholesteryl ester, an unknown compound 389.21933 m/z (under negative ionisation), an unknown compound 886.71063 m/z (under negative ionisation), citric acid and MG(0:0/18:3(6Z,9Z,12Z)/0:0.

In preferred embodiments, the method of the present invention comprises the steps of:
(i) determining the level of nine biomarkers in a sample from the subject;
(ii) comparing the level of said nine biomarkers with the level of said nine biomarkers in a control sample to determine whether Mycobacterium bovis is present in the subject;
   wherein the biomarkers consist of dihydro-leukotriene B4, 3-hydroxyisovaleric acid, sphingomyelin [SM(d18:2(4E,14Z)/24:0)], sphingomyelin [SM(d18:1/24:1(15Z))], 18:3 cholesteryl ester, an unknown compound 389.21933 m/z (under negative ionisation), an unknown compound 886.71063 m/z (under negative ionisation), citric acid and MG(0:0/18:3(6Z,9Z,12Z)/0:0.

The present inventors have found that using these biomarkers, in combination, offers a “signature” or “diagnostic fingerprint” which allows the highly accurate identification of bovine tuberculosis. Individually, each biomarker has accuracies of greater than 0.77 (AUC value) and by utilising a combination of these biomarkers a highly accurate diagnosis can be made with accuracies of greater than 0.99 (AUC value).

In embodiments of the present invention, the method may be for distinguishing between subjects both infected with and vaccinated against Mycobacterium bovis and subjects vaccinated against Mycobacterium bovis.

Thus, additionally the present inventors have shown that a number of the metabolite biomarkers identified can distinguish between subjects infected with Mycobacterium bovis and those which have be vaccinated, something which has, to date, been difficult.

In such embodiments, the one or more biomarkers are preferably selected from: dihydro-leukotriene B4, pyrocatechol, MG(0:0/18:3(6Z,9Z,12Z)/0:0), N-(docosanoyl)-heptadecasphing-4-enine-1-phosphocholine, 3-hydroxyisovaleric acid, sphingomyelin [SM(d18:2(4E,14Z)/24:0)], 18:3 cholesteryl ester, citric acid, an unknown compound 389.21933 m/z (under negative ionisation) and an unknown compound 359.22107 m/z (under negative ionisation).

Therefore, the present invention provides a method for determining whether a vaccinated subject is infected with Mycobacterium bovis, the method comprising the steps of:
(i) determining the level of one or more biomarkers in a sample from the subject;
(ii) comparing the level of said one or more biomarkers with the level of said one or more biomarkers in a control sample to determine whether the subject is infected with Mycobacterium bovis;
   wherein the one or more biomarkers are selected from: dihydro-leukotriene B4, pyrocatechol, MG(0:0/18:3(6Z,9Z,12Z)/0:0), N-(docosanoyl)-heptadecasphing-4-enine-1-phosphocholine, 3-hydroxyisovaleric acid, sphingomyelin [SM(d18:2(4E,14Z)/24:0)], 18:3 cholesteryl ester, citric acid, an unknown compound 389.21933 m/z (under negative ionisation) and an unknown compound 359.22107 m/z (under negative ionisation).

When we refer to a “vaccinated subject” we refer to a subject who is vaccinated against Mycobacterium bovis. The present inventors have found that these specific biomarkers are able to differentiate between subjects both infected with and vaccinated against Mycobacterium bovis and subjects vaccinated against Mycobacterium bovis (and not infected).

In embodiments of the invention, the method may comprise determining the level of two, three, four, five, six, seven, eight, nine or ten of the following biomarkers: dihydro-leukotriene B4, pyrocatechol, MG(0:0/18:3(6Z,9Z,12Z)/0:0), N-(docosanoyl)-heptadecasphing-4-enine-1-phosphocholine, 3-hydroxyisovaleric acid, sphingomyelin [SM(d18:2(4E,14Z)/24:0)], 18:3 cholesteryl ester, citric acid, an unknown compound 389.21933 m/z (under negative ionisation) and an unknown compound 359.22107 m/z (under negative ionisation).

In embodiments in which the method involves determining the level of ten biomarkers in a sample from the subject, the biomarkers may consist of dihydro-leukotriene B4, pyrocatechol, MG(0:0/18:3(6Z,9Z,12Z)/0:0), N-(docosanoyl)-heptadecasphing-4-enine-1-phosphocholine, 3-hydroxyisovaleric acid, sphingomyelin [SM(d18:2(4E,14Z)/24:0)], 18:3 cholesteryl ester, citric acid, an unknown compound 389.21933 m/z (under negative ionisation) and an unknown compound 359.22107 m/z (under negative ionisation).

In preferred embodiments, the method comprises the steps of:
(i) determining the level of ten biomarkers in a sample from the subject;
(ii) comparing the level of said ten biomarkers with the level of said ten biomarkers in a control sample to determine whether the subject is infected with Mycobacterium bovis;
   wherein the biomarkers consist of dihydro-leukotriene B4, pyrocatechol, MG(0:0/18:3(6Z,9Z,12Z)/0:0), N-(docosanoyl)-heptadecasphing-4-enine-1-phosphocholine, 3-hydroxyisovaleric acid, sphingomyelin [SM(d18:2(4E,14Z)/24:0)], 18:3 cholesteryl ester, citric acid, an unknown compound 389.21933 m/z (under negative ionisation) and an unknown compound 359.22107 m/z (under negative ionisation).

The present inventors have found that using these biomarkers, in combination, offers a “signature” or “diagnostic fingerprint” which allows the highly accurate differentiation between subjects infected with and vaccinated against Mycobacterium bovis and subjects which have been vaccinated and are not infected with Mycobacterium bovis. Individually, each biomarker has accuracies of greater than 0.69 (AUC value) and by utilising a combination of these biomarkers a highly accurate diagnosis can be made with accuracies of greater than 0.99 (AUC value).

In embodiments of the present invention, the biomarkers comprise or consist of dihydro-leukotriene B4, MG(0:0/18:3(6Z,9Z,12Z)/0:0), 3-hydroxyisovaleric acid, sphingomyelin [SM(d18:2(4E,14Z)/24:0)], 18:3 cholesteryl ester, citric acid and unknown compound 389.21933 (under negative ionisation). The present inventors have found that these biomarkers, in combination, have a combined AUCs in excess of 0.99 for both discriminating infected and uninfected cattle, and differentiating infected from vaccinated samples. Crucially, this seven-feature panel can identify infection in vaccinated animals, where protection appears to have failed.

The method of the present invention involves determining the level of one or more biomarkers in a sample. The skilled person will appreciate that there are a number of ways in which these biomarker levels can be determined.

For example, the biomarker level may be determined using mass spectrometry, for example high resolution mass spectrometry (HR-MS), gas chromatography time-of-flight mass spectrometry (GC-MS), flow infusion electrospray high resolution mass spectrometry (FIE-HRMS) or liquid chromatography-electrospray mass spectrometry (LC-MS).

GC-MS involves linking a gas chromatograph with a mass spectrometer. The gas chromatograph utilizes a capillary column where the chemical properties between the sampled chemicals a mixture and their relative affinity for the stationary phase of the column will result in their separation along the column. This provides “retention time”, information. The chemicals then enter the mass spectrometer which will show mass-to-charge ratios.

LC-MS links liquid chromatography (LC or High-Performance LC [HPLC]) with a mass spectrometer. The LC part physically separates chemicals between a liquid mixture of two immiscible phases, i.e., stationary and mobile. The chemicals then enter the mass spectrometer which will show mass-to-charge ratios.

In flow infusion, samples are injected directly into a solvent (usually methanol-water) line leading to a mass spectrometer.

Alternatively, the biomarker level could be determined using NMR, enzymatic assays (e.g. enzymatic reaction followed by colorimetric detection) or immunoassays (i.e. antibody binding based assays).

The most well-established immunoassay is the enzyme-linked immunosorbent assay (ELISA). The most used ELISA technique is the sandwich ELISA which measures the antigen using a capture (often associated within the well of a 96 well plate to allow high-throughput screening) and a detection antibody. Monoclonal or polyclonal antibodies can be used in sandwich or competitive ELISA systems. Other immunoassay systems include lateral flow or flow through systems. The use of such immunoassays for detecting the biomarker level would allow the method to be used as a Point of Care (POC) test, for example a POC device, allowing the presence of Mycobacterium bovis to be determined in locations away from a laboratory, for example at a farm or remote location.

The step of determining the biomarker level (and comparing the biomarker level with the biomarker level in a control sample) may be performed using a POC test device, for example the device described below. For example, the step of determining the biomarker level (and comparing the biomarker level with the biomarker level in a control sample) may be performed using a flow through device or a lateral flow device. In embodiments, the step of determining the biomarker level (and comparing the biomarker level with the biomarker level in a control sample) may be performed using a lateral flow device, for example the device described below.

The term “biomarker level” as used herein is used to mean the amount of biomarker present in the samples. As will be appreciated by the skilled person, this would be standardised with a set volume of sample used in the assay and the presence of internal controls, for example the detection of serum albumin levels (so that biomarker levels are determined relative to this established standard).

Step (ii) of the method of the present invention involves comparing the level of said one or more biomarkers with the level of said one or more biomarkers in a control sample to determine whether Mycobacterium bovis is present in the subject.

As will be appreciated by the skilled person, the origin of the control sample will depend upon the particular subject being tested. However, the control sample may be obtained from an age-matched subject and/or a subject of the same sex. Furthermore, since the comparison of biomarker levels may be used to determine whether Mycobacterium bovis is present in a subject, the control sample may be derived, for example, from a subject in which Mycobacterium bovis is not present. Since the comparison of biomarker levels may subsequently be used to determine whether a subject has bovine tuberculosis (including subclinical bovine tuberculosis), the control sample by be derived from a subject who does not have bovine tuberculosis. Since the comparison of biomarker levels may be used to determine whether Mycobacterium bovis is present in a vaccinated individual, the control sample may be derived from a subject who is vaccinated against Mycobacterium bovis.

In the method of the present invention the biomarker level in a sample from a subject is compared with the biomarker level in a sample from a control. As will be appreciated by the skilled person, in the comparison step, the level of a biomarker in the sample from the subject is compared with a corresponding biomarker level in the control sample (e.g. in embodiments in which the metabolite dihydro-leukotriene B4 is utilised, the level of dihydro-leukotriene B4 in the sample is compared with the level of dihydro-leukotriene B4 in the control).

In embodiments of the invention, a biomarker level in the sample from the subject which is higher or lower than the biomarker level in the control sample indicates that Mycobacterium bovis is present in the subject.

In embodiments of the invention in which the one or more biomarkers are selected from dihydro-leukotriene B4, sphingomyelin [SM(d18:2(4E,14Z)/24:0)], sphingomyelin [SM(d18:1/24:1(15Z))],18:3 cholesteryl ester, unknown compound 866.71063 m/z (under negative ionisation) or MG(0:0/18:3(6Z,9Z,12Z)/0:0), a biomarker level in the sample from the subject which is higher than the biomarker level in the control sample indicates that Mycobacterium bovis is present in the subject.

In embodiments of the invention in which the one or more biomarkers are selected from: 3-hydroxyisovaleric acid, unknown compound 389.21933 m/z (under negative ionisation) and citric acid, a biomarker level in the sample from the subject which is lower than the biomarker level in the control sample indicates that Mycobacterium bovis is present in the subject.

In embodiments of the invention in which the one or more biomarkers include dihydro-leukotriene B4, an unknown compound 359.22107 m/z (under negative ionisation), 3-hydroxyisovaleric acid, citric acid and 18:3 cholesteryl ester, a biomarker level in the sample which is higher than the biomarker level in the control sample indicates that the subject is infected with Mycobacterium bovis, in vaccinated individuals.

In embodiments of the invention in which the one or more biomarkers are selected from: pyrocatechol, MG(0:0/18:3(6Z,9Z,12Z)/0:0), N-(docosanoyl)-heptadecasphing-4-enine-1-phosphocholine, unknown compound 389.21933 m/z (under negative ionisation), sphingomyelin [SM(d18:2(4E,14Z)/24:0)], a biomarker level in the sample which is lower than the biomarker level in the control sample indicates that the subject is infected with Mycobacterium bovis, in vaccinated individuals.

As will be appreciated by the skilled person, when referring to a higher or lower biomarker level in a sample from the subject compared to a control sample, we mean a biomarker level which is significantly higher (at least two-fold higher) or significantly lower (at least 0.5-fold lower) than the biomarker level in a control sample.

In embodiments in which the biomarker level in the sample from the subject is higher than the biomarker level in the control sample, preferably the biomarker level in the sample from the subject is at least two-fold higher than in the control sample. More preferably, the biomarker level in the sample from the subject is at least three-fold, four-fold or five-fold higher than in the control sample. More preferably, the biomarker level in the sample from the subject is at least six-fold, seven-fold, eight-fold, nine-fold or ten-fold higher than in the control sample.

In embodiments in which the biomarker level in the sample from the subject is lower than the biomarker level in the control sample, preferably the biomarker level in the sample from the subject is at least two-fold lower than in the control sample. More preferably, the biomarker level in the sample from the subject is at least three-fold, four-fold or five-fold lower than in the control sample. More preferably, the biomarker level in the sample from the subject is at least six-fold, seven-fold, eight-fold, nine-fold or ten-fold lower than in the control sample.

The present invention provides a method for determining the presence of Mycobacterium bovis in a subject. As discussed above, Mycobacterium bovis causes bovine tuberculosis, a chronic disease which primarily affects cattle, although cases have also been described in a variety of other mammals including, for example, deer, goats, pigs, cats, dogs, badgers and humans.

In embodiments of the present invention, the subject is an animal, for example, a cattle, deer, goat, pig, cat, dog or badger. Alternatively, the subject may be a human. In preferred embodiments, the subject is domestic cattle, which includes beef cattle and dairy cows for example.

The method of the present invention may further comprise providing a sample from a subject. In such embodiments the method of the present invention comprises the steps of:
(i) providing a sample from a subject;
(ii) determining the level of one or more biomarkers in the sample; and
(iii) comparing the level of said one or more biomarkers with the level of said one or more biomarkers in a control sample to determine whether Mycobacterium bovis is present in the subject;
   wherein the one or more biomarkers are selected from: dihydro-leukotriene B4, 3-hydroxyisovaleric acid, sphingomyelin [SM(d18:2(4E,14Z)/24:0)], sphingomyelin [SM(d18:1/24:1(15Z))], 18:3 cholesteryl ester, an unknown compound 389.21933 m/z (under negative ionisation), an unknown compound 886.71063 m/z (under negative ionisation), citric acid, MG(0:0/18:3(6Z,9Z,12Z)/0:0), pyrocatechol, N-(docosanoyl)-heptadecasphing-4-enine-1-phosphocholine and an unknown compound 359.22107 m/z (under negative ionisation).

Features of the abovementioned methods described herein apply equally to this method.

The present invention comprises determining the level of one or more biomarkers in a sample from the subject. As will be appreciated by the skilled person, the sample may comprise a biological sample from the subject. The biological sample may have been obtained from a bodily fluid of the subject (e.g. cattle). The biological sample, may include, for example, blood and blood components (e.g. serum), mucus, saliva, urine, milk, vomit, faeces, sweat, semen, vaginal secretion, tears, pus or milk. Preferably, the biological sample is a blood sample. As will be appreciated by the skilled person, the method of the present invention is therefore an in vitro method for determining the presence of Mycobacterium bovis in a subject, the method being carried out on a sample provided from a subject.

In a further aspect of the invention there is provided a method for determining the presence of Mycobacterium bovis in a subject, the method comprising the steps of:
(i) obtaining a sample from the subject;
(ii) determining the level of one or more biomarkers in the sample;
(iii) comparing the level of said one or more biomarkers with the level of said one or more biomarkers in a control sample to determine whether Mycobacterium bovis is present in the subject;
   wherein the one or more biomarkers are selected from: dihydro-leukotriene B4, 3-hydroxyisovaleric acid, sphingomyelin [SM(d18:2(4E,14Z)/24:0)], sphingomyelin [SM(d18:1/24:1(15Z))], 18:3 cholesteryl ester, an unknown compound 389.21933 m/z (under negative ionisation), an unknown compound 886.71063 m/z (under negative ionisation), citric acid, MG(0:0/18:3(6Z,9Z,12Z)/0:0), pyrocatechol, N-(docosanoyl)-heptadecasphing-4-enine-1-phosphocholine and an unknown compound 359.22107 m/z (under negative ionisation).

The method of obtaining the sample from the subject will depend on the sample type and subject. In embodiments in which the sample is a blood sample, the step of obtaining the sample may comprise using a needle and syringe or a double-pointed needle and vacutainer tube.

There is also provided a method for determining the presence of bovine tuberculosis in a subject, determining progression of bovine tuberculosis or assessing response to therapy of a subject with bovine tuberculosis. The method comprises the steps of:
(i) determining the level of one or more biomarkers in a sample from the subject;
(ii) comparing the level of said one or more biomarkers with the level of said one or more biomarkers in a control sample to determine whether the subject has bovine tuberculosis, has bovine tuberculosis which is progressing, or is responding to therapy for bovine tuberculosis;
   wherein the one or more biomarkers are selected from: dihydro-leukotriene B4, 3-hydroxyisovaleric acid, sphingomyelin [SM(d18:2(4E,14Z)/24:0)], sphingomyelin [SM(d18:1/24:1(15Z))], 18:3 cholesteryl ester, an unknown compound 389.21933 m/z (under negative ionisation), an unknown compound 886.71063 m/z (under negative ionisation), citric acid, MG(0:0/18:3(6Z,9Z,12Z)/0:0), pyrocatechol, N-(docosanoyl)-heptadecasphing-4-enine-1-phosphocholine and an unknown compound 359.22107 m/z (under negative ionisation).

There is also provided a method for determining the presence of bovine tuberculosis in a subject, determining progression of bovine tuberculosis or assessing response to therapy of a subject with bovine tuberculosis. The method comprises the steps of:
(i) obtaining a sample from the subject;
(ii) determining the level of one or more biomarkers in a sample from the subject;
(iii) comparing the level of said one or more biomarkers with the level of said one or more biomarkers in a control sample to determine whether the subject has bovine tuberculosis, has bovine tuberculosis which is progressing, or is responding to therapy for bovine tuberculosis;
   wherein the one or more biomarkers are selected from: dihydro-leukotriene B4, 3-hydroxyisovaleric acid, sphingomyelin [SM(d18:2(4E,14Z)/24:0)], sphingomyelin [SM(d18:1/24:1(15Z))], 18:3 cholesteryl ester, an unknown compound 389.21933 m/z (under negative ionisation), an unknown compound 886.71063 m/z (under negative ionisation), citric acid, MG(0:0/18:3(6Z,9Z,12Z)/0:0), pyrocatechol, N-(docosanoyl)-heptadecasphing-4-enine-1-phosphocholine and an unknown compound 359.22107 m/z (under negative ionisation).

In embodiments where the method is being used to determine whether bovine tuberculosis is progressing, the method may comprise monitoring over time to determine whether bovine tuberculosis has progressed. In such an embodiment, an initial biomarker level (as described above) may be compared with a biomarker level in a sample obtained later in time. The biomarker level(s) obtained may change over time, to be further removed (either higher or lower) from a control biomarker level. Alternatively, in examples where multiple biomarkers are utilised, for example six biomarkers, in an initial analysis only three of the six biomarkers may be indicative of bovine tuberculosis. In the later analysis an increased number of biomarkers may be indicative of bovine tuberculosis, which may indicate that bovine tuberculosis is progressing. This could be done even if a subject had been vaccinated (and the vaccination proved to be either fully or partially ineffective) and subsequently infected with bovine tuberculosis. As discussed above, the present inventors have shown that in a controlled experiment comparing vaccination and infection with BCG vaccine, infection changed the levels of dihydro-leukotriene B4, pyrocatechol, MG(0:0/18:3(6Z,9Z,12Z)/0:0), N-(docosanoyl)-heptadecasphing-4-enine-1-phosphocholine, 3-hydroxyisovaleric acid, sphingomyelin [SM(d18:2(4E,14Z)/24:0)], 18:3 cholesteryl ester, citric acid, an unknown compound 389.21933 m/z (under negative ionisation) and an unknown compound 359.22107 m/z (under negative ionisation) but no change was observed in the vaccinated population until infected with bovine tuberculosis. Dihydro-leukotriene B4, pyrocatechol, MG(0:0/18:3(6Z,9Z,12Z)/0:0), N-(docosanoyl)-heptadecasphing-4-enine-1-phosphocholine, 3-hydroxyisovaleric acid, sphingomyelin [SM(d18:2(4E,14Z)/24:0)], 18:3 cholesteryl ester, citric acid, an unknown compound 389.21933 m/z (under negative ionisation) and an unknown compound 359.22107 m/z (under negative ionisation) were therefore shown to have the capability to differentiate bovine tuberculosis infected, from vaccinated cattle.

In embodiments where the method is being used to determine whether a subject is responding to therapy, the method may comprise monitoring over time to determine whether a subject is responding to therapy. In such an embodiment, an initial biomarker analysis (which may be before therapy has commenced) may be compared with one or more biomarker analyses undertaken on samples obtained later in time (for example after therapy has commenced). The biomarker levels may change over time, to be further removed (either higher or lower) from a control biomarker level. Alternatively, in examples where multiple biomarkers are utilised, for example six biomarkers, in an initial analysis, six out of six biomarker levels may be indicative of bovine tuberculosis. In a later analysis, fewer biomarkers may be indicative of bovine tuberculosis, which may indicate that the subject is responding to therapy. Alternatively, in a later analysis, the number of biomarkers indicative of bovine tuberculosis may remain the same or increase, which may indicate that a subject is not responding to therapy.

In the case of humans, the method of the present invention may also be used to determine whether a drug is effective at treating bovine tuberculosis, in a similar manner to that described above in relation to determining whether a subject is responding to therapy. The use of “effective” is used to indicate that a treatment reduces or alleviates signs or symptoms of bovine tuberculosis, improves the clinical course of the disease, decreases the number or severity of exacerbations or reduces any other objective or subjective indicia of the disease. The method of the present invention can be used to determine whether drugs used to treat bovine tuberculosis, in addition to other drugs developed to treat bovine tuberculosis are effective. The use of the method of the present invention to determine whether a drug is effective at treating bovine tuberculosis is currently only relevant to human subjects as all other animal species testing positive for bovine tuberculosis are culled. However, it is possible that in the future, the management of animals with bovine tuberculosis may be different (for example, endangered and protected animals may not be culled, but vaccinated and treated) and the method of the present invention may be used to determine whether a drug is effective at treating bovine tuberculosis in animals.

Since a diagnosis of a disease is often not based on the results of a single test alone, the method of the present invention may be used to determine whether a subject is more likely than not to have bovine tuberculosis based on comparison of one or more biomarker levels with a control biomarker level. Thus, for example, a subject with a putative diagnosis of bovine tuberculosis may be diagnosed as being “more likely” or “less likely” to have bovine tuberculosis in light of the information provided by the method of the present invention. The present invention may therefore be used to assist a clinician/veterinarian with the diagnosis of bovine tuberculosis.

The method of the present invention may, in certain embodiments, comprise detecting other signs or symptoms of bovine tuberculosis, conducting clinical tests of bovine tuberculosis and/or measuring other bovine tuberculosis markers, for example other alternative biomarkers.

As will be appreciated by the skilled person, the above description is not limited to making an initial identification (or diagnosis) of bovine tuberculosis in a subject but is also applicable to confirming a provisional diagnosis of bovine tuberculosis or “ruling out” such a diagnosis.

The present invention may also be used to determine a suitable treatment for a subject, depending on whether testing indicates that they are infected with Mycobacterium bovis/have bovine tuberculosis. The use of the method of the present invention to determine a suitable treatment for a subject is currently only relevant to humans as all other animal species testing positive of bovine tuberculosis are culled. However, it is possible that in the future, the management of animals with bovine tuberculosis may be different (for example, endangered and protected animals may not be culled, but vaccinated and treated). In such embodiments, the method of the present invention may comprise a step of determining a suitable treatment for the subject, if the subject has is infected with Mycobacterium bovis/has bovine tuberculosis.

The present invention also provides an immunological capture device for detecting Mycobacterium bovis in a subject, the device comprising a substrate carrying capture antibodies to one or more of the following biomarkers: dihydro-leukotriene B4, 3-hydroxyisovaleric acid, sphingomyelin [SM(d18:2(4E,14Z)/24:0)], sphingomyelin [SM(d18:1/24:1(15Z))], 18:3 cholesteryl ester, an unknown compound 389.21933 m/z (under negative ionisation), an unknown compound 886.71063 m/z (under negative ionisation), citric acid, MG(0:0/18:3(6Z,9Z,12Z)/0:0), pyrocatechol, N-(docosanoyl)-heptadecasphing-4-enine-1-phosphocholine and an unknown compound 359.22107 m/z (under negative ionisation).

The present invention also provides an immunological capture device for detecting bovine tuberculosis in a subject, the device comprising a substrate carrying capture antibodies to one or more of the following biomarkers: dihydro-leukotriene B4, 3-hydroxyisovaleric acid, sphingomyelin [SM(d18:2(4E,14Z)/24:0)], sphingomyelin [SM(d18:1/24:1(15Z))], 18:3 cholesteryl ester, an unknown compound 389.21933 m/z (under negative ionisation), an unknown compound 886.71063 m/z (under negative ionisation), citric acid, MG(0:0/18:3(6Z,9Z,12Z)/0:0), pyrocatechol, N-(docosanoyl)-heptadecasphing-4-enine-1-phosphocholine and an unknown compound 359.22107 m/z (under negative ionisation).

In embodiments, the substrate carries capture antibodies to one or more of the following biomarkers: dihydro-leukotriene B4, 3-hydroxyisovaleric acid, sphingomyelin [SM(d18:2(4E,14Z)/24:0)], sphingomyelin [SM(d18:1/24:1(15Z))], 18:3 cholesteryl ester, an unknown compound 389.21933 m/z (under negative ionisation), an unknown compound 886.71063 m/z (under negative ionisation), citric acid and MG(0:0/18:3(6Z,9Z,12Z)/0:0.

In embodiments, the substrate carries capture antibodies to dihydro-leukotriene B4, pyrocatechol, MG(0:0/18:3(6Z,9Z,12Z)/0:0), N-(docosanoyl)-heptadecasphing-4-enine-1-phosphocholine, 3-hydroxyisovaleric acid, sphingomyelin [SM(d18:2(4E,14Z)/24:0)], 18:3 cholesteryl ester, citric acid, an unknown compound 389.21933 m/z (under negative ionisation) and an unknown compound 359.22107 m/z (under negative ionisation). Such a device can be particularly useful in distinguishing between subjects both infected with and vaccinated against Mycobacterium bovis and subjects vaccinated against Mycobacterium bovis.

In embodiments, the substrate carries capture antibodies to all listed biomarkers.

The term “immunological capture device” as described herein is used to describe an immunoassay device which can be used to measure the presence of a biomarker in a sample through the use of an antibody. The antibody would be specific to the biomarker of interest, such that the antibody could “capture” the biomarker, through binding, if the biomarker is present. The antibodies may therefore be described as “capture antibodies”.

The device comprises antibodies to one or more of the following biomarkers: dihydro-leukotriene B4, 3-hydroxyisovaleric acid, sphingomyelin [SM(d18:2(4E,14Z)/24:0)], sphingomyelin [SM(d18:1/24:1(15Z))], 18:3 cholesteryl ester, an unknown compound 389.21933 m/z (under negative ionisation), an unknown compound 886.71063 m/z (under negative ionisation), citric acid, MG(0:0/18:3(6Z,9Z,12Z)/0:0), pyrocatechol, N-(docosanoyl)-heptadecasphing-4-enine-1-phosphocholine and an unknown compound 359.22107 m/z (under negative ionisation).

In embodiments, the device comprises antibodies to two, three, four, five, six, seven, eight or nine of the following biomarkers: dihydro-leukotriene B4, 3-hydroxyisovaleric acid, sphingomyelin [SM(d18:2(4E,14Z)/24:0)], sphingomyelin [SM(d18:1/24:1(15Z))], 18:3 cholesteryl ester, an unknown compound 389.21933 m/z (under negative ionisation), an unknown compound 886.71063 m/z (under negative ionisation), citric acid and MG(0:0/18:3(6Z,9Z,12Z)/0:0.

In embodiments, the device comprises antibodies to two, three, four, five, six, seven, eight, nine or ten of the following biomarkers: dihydro-leukotriene B4, pyrocatechol, MG(0:0/18:3(6Z,9Z,12Z)/0:0), N-(docosanoyl)-heptadecasphing-4-enine-1-phosphocholine, 3-hydroxyisovaleric acid, sphingomyelin [SM(d18:2(4E,14Z)/24:0)], 18:3 cholesteryl ester, citric acid, an unknown compound 389.21933 m/z (under negative ionisation) and an unknown compound 359.22107 m/z (under negative ionisation).

In embodiments, the device comprises antibodies to two, three, four, five, six or seven of the following biomarkers: dihydro-leukotriene B4, MG(0:0/18:3(6Z,9Z,12Z)/0:0), 3-hydroxyisovaleric acid, sphingomyelin [SM(d18:2(4E,14Z)/24:0)], 18:3 cholesteryl ester, citric acid and unknown compound 389.21933 (under negative ionisation).

The substrate may be any suitable surface which can carry an antibody. For example, the substrate may be plastic, for example a plate (e.g. a multi-well plate). Alternatively, the substrate may be a porous substrate. The porous substrate may be any material which allows another medium to pass through it. Any suitable porous substrate could be used, for example a woven material, or a cellulosic material.

The substrate carries the capture antibodies. The antibodies may be carried within the substrate or on the surface of the substrate. The antibodies may form a chemical interaction with the surface of the substrate. The antibodies may be bound to the substrate.

In embodiments, the device is a lateral flow device. Alternatively, the device may be a flow through device or an ELISA device.

Advantageously the device is a rapid, simple, non-invasive diagnostic providing quick diagnosis of bovine tuberculosis. It is easily accessible to farmers, for example, to improve detection and control of bovine tuberculosis. The device allows farmers to monitor bovine tuberculosis and specifically target treatment to those animals with bovine tuberculosis. A reliable on-farm diagnostic kit will help prevent disease, particularly the spread of disease, and increase the efficiency of livestock production.

Lateral flow and flow through devices are particularly advantageous in that they can be used remotely to obtain rapid results in a simple manner.

A sample to be tested (i.e. a sample from the subject) may be applied to the immunological capture device, for example to the substrate of the immunological capture device.

A second antibody may also be applied the substrate of the immunological capture device. The second antibody may be specific to the biomarkers or to the capture antibodies (i.e. the antibodies carried on the substrate). The second antibody may have coloured particles attached. The coloured particles may be covalently linked to the second antibody. The coloured particles may be cellulose beads or plastic microparticles, for example. In alternative embodiments the second antibody may be linked to an enzyme, via bio-conjugation, for example. In such embodiments, a composition comprising a substrate which undergoes a colour change upon reaction with the enzyme, indicating the presence of the enzyme, may be added to the device during use. Suitable enzymes and compositions will be well known to the skilled person.

In embodiments in which the second antibody is specific to the biomarkers, binding of the second antibody to the biomarkers bound to the capture antibodies results in a colour change. The presence of the biomarkers can therefore be detected by a colour change.

In embodiments in which the second antibody is specific to the capture antibodies, binding of the second antibody to the capture antibody may take place when the biomarker is not present. In such an embodiment, binding of the second antibody to the capture antibodies, results in a colour change. The absence of the biomarkers can therefore be detected by a colour change.

A reader, for example a lateral flow reader may be used to quantify the colour intensity.

In embodiments in which the device is a lateral flow or flow through device, the device may further comprise a control line. Colouring of the control line indicates successful completion of the test.

In embodiments in which the device is a lateral flow or flow through device, the device may further comprise a test line. The test line may comprise capture antibodies to the one or more biomarkers.

Colouring of the test line indicates the presence or absence of the biomarkers in the sample (as discussed above). Comparison of the coloured intensity of the control line and test line can be used to indicate the biomarker level in the sample and therefore whether the sample is from a subject in which Mycobacterium bovis or bovine tuberculosis is present. A reader, for example a lateral flow reader may be used to quantify the coloured intensity of the control and test lines.

In embodiments, the device may further comprise a housing. The substrate may be positioned within the housing.

The device can be used to detect biomarkers in blood serum and whole blood samples, for example.

Preferably the device provides a test result in 60 minutes or less from test initiation. The device may provide a test result in 30 minutes or less, 20 minutes or less or 10 minutes or less from test initiation.

The device may be used in the method of the invention.

The present invention also provides a kit for determining the presence of Mycobacterium bovis or bovine tuberculosis in a subject, the kit comprising:
(i) an immunological capture device comprising a substrate carrying capture antibodies to one or more of the following biomarkers: dihydro-leukotriene B4, 3-hydroxyisovaleric acid, sphingomyelin [SM(d18:2(4E,14Z)/24:0)], sphingomyelin [SM(d18:1/24:1(15Z))], 18:3 cholesteryl ester, an unknown compound 389.21933 m/z (under negative ionisation), an unknown compound 886.71063 m/z (under negative ionisation), citric acid, MG(0:0/18:3(6Z,9Z,12Z)/0:0), pyrocatechol, N-(docosanoyl)-heptadecasphing-4-enine-1-phosphocholine and an unknown compound 359.22107 m/z (under negative ionisation); and
(ii) a second antibody, wherein the second antibody is specific to the one or more biomarkers or to the capture antibodies.

The device and second antibody of the kit are described above in relation to the immunological capture device of the present invention. The kit may be used in the method of the invention.

The present invention also provides the use of one or more biomarkers for detecting Mycobacterium bovis in a subject, the one or more biomarkers being selected from: dihydro-leukotriene B4, 3-hydroxyisovaleric acid, sphingomyelin [SM(d18:2(4E,14Z)/24:0)], sphingomyelin [SM(d18:1/24:1(15Z))], 18:3 cholesteryl ester, an unknown compound 389.21933 m/z (under negative ionisation), an unknown compound 886.71063 m/z (under negative ionisation), citric acid, MG(0:0/18:3(6Z,9Z,12Z)/0:0), pyrocatechol, N-(docosanoyl)-heptadecasphing-4-enine-1-phosphocholine and an unknown compound 359.22107 m/z (under negative ionisation).

The present invention also provides the use of one or more biomarkers for detecting bovine tuberculosis in a subject, the one or more biomarkers being selected from: dihydro-leukotriene B4, 3-hydroxyisovaleric acid, sphingomyelin [SM(d18:2(4E,14Z)/24:0)], sphingomyelin [SM(d18:1/24:1(15Z))], 18:3 cholesteryl ester, an unknown compound 389.21933 m/z (under negative ionisation), an unknown compound 886.71063 m/z (under negative ionisation), citric acid, MG(0:0/18:3(6Z,9Z,12Z)/0:0), pyrocatechol, N-(docosanoyl)-heptadecasphing-4-enine-1-phosphocholine and an unknown compound 359.22107 m/z (under negative ionisation).

The described and illustrated embodiments are to be considered as illustrative and not restrictive in character, it being understood that only the preferred embodiments have been shown and described and that all changes and modifications that come within the scope of the inventions as defined in the claims are desired to be protected.

The optional features set out herein may be used either individually or in combination with each other where appropriate and particularly in the combinations as set out in the accompanying claims. The optional features for each aspect or exemplary embodiment of the invention as set out herein are also to be read as applicable to any other aspect or exemplary embodiments of the invention, where appropriate. In other words, the skilled person reading this specification should consider the optional features for each exemplary embodiment of the invention as interchangeable and combinable between different exemplary embodiments.

It should be understood that while the use of words such as “preferable”, “preferably”, “preferred” or “more preferred” in the description suggest that a feature so described may be desirable, it may nevertheless not be necessary and embodiments lacking such a feature may be contemplated as within the scope of the invention as defined in the appended claims. In relation to the claims, it is intended that when words such as “a,” “an,” or “at least one,” are used to preface a feature there is no intention to limit the claim to only one such feature unless specifically stated to the contrary in the claim.

### DETAILED DESCRIPTION OF THE INVENTION

The present invention will now be further described with reference to the following figures which show:

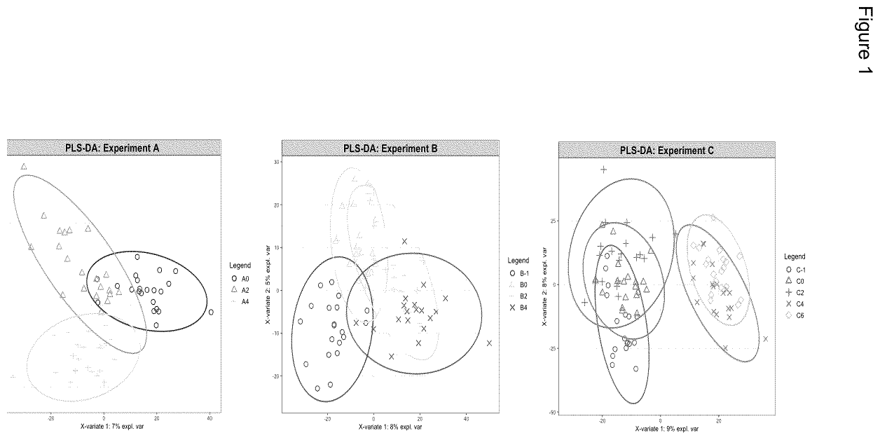

FIG. 1: Partial least squares-discriminant analysis (PLSDA) plots of three individual experiments (A. B and C) where cattle were experimentally infected with M. bovis. Samples were obtained one week before infection (−1), at infection (0) and 2, 4, and 6 weeks after infection. The sampling times varied between experiments.

Metabolomic assessments were undertaken of three separate experiments where cattle were infected with M. bovis. Each experiment individually to indicate metabolomic changes over the course of M. bovis infection: Legends: experiments A, B and C, numbers refer to weeks prior to (−1), at (0) or subsequent to experimental infection with M. bovis. In each case, the plasma metabolome showed a separation between pre- and post-infection samples at 4 weeks.

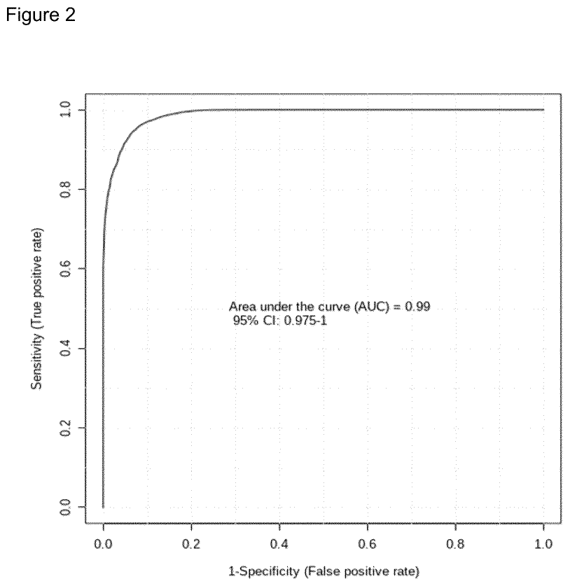

FIG. 2: Receiver Operator Curve analysis using the biomarker fingerprint.

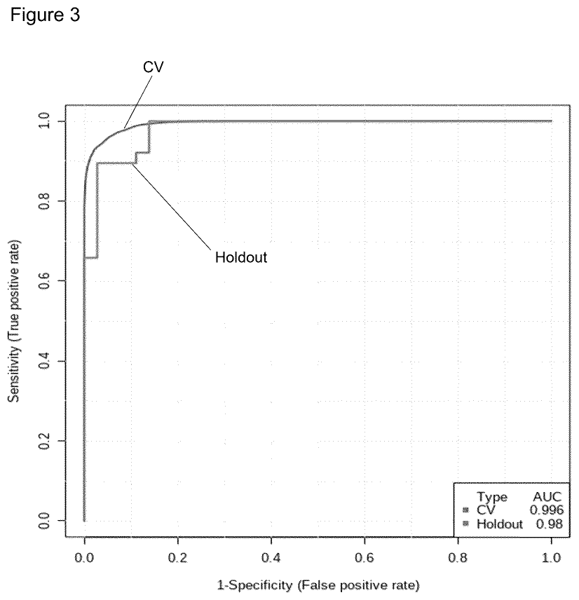

FIG. 3: Five feature ROC analysis of groups A and B (CV—cross-validation) compared to C as validation group.

As discussed above, the method of the present invention allows the presence of Mycobacterium bovis in a subject to be detected. The method of the present invention therefore allows the presence of bovine tuberculosis, for example subclinical bovine tuberculosis, to be detected. The present inventors undertook significant investigation to develop the method of the present invention and identified a number of biomarker metabolites, the level of which is significantly altered in subjects in which Mycobacterium bovis/bovine tuberculosis is present. The present inventors have therefore identified a subset of biomarkers which can be used to quickly and accurately identify animals affected by bovine tuberculosis, so that they can be treated accordingly.

In addition, the present inventors identified a subset of biomarkers which can be used to differentiate between subjects which have been vaccinated and those which have been vaccinated and are infected with Mycobacterium bovis.

### Materials and Methods

### Experimental Infection

Three experimental infections, referred to as A, B and C, took place at different times over a 12-month period. Male entire Holstein-Friesian calves aged approximately six months of age were sourced from bovine tuberculosis free herds in the low-risk area of England. Following acclimatisation calves were infected by endobronchial inoculation with approximately 10,000 CFU of M. bovis strain AF 2122/97. Blood samples were collected at several time points, both prior to and following infection, the number of calves and sampling points are summarised in Table 1. Blood samples were collected by jugular venepuncture into heparinised falcon tubes, with the 0-week sample collected prior to infection. Plasma was harvested and frozen.

TABLE 1
Number of animals and timepoints used
in each experimental infection

| Infection experiment | A | B | C |
|---|---|---|---|
| Number of animals | 22 | 20 | 19 |
| Time points sampled | 0, 2, 4 | −1, 0, 2, 4 | −1, 0, 2, 4, 6 |
| (weeks relative to | | | |
| infection) | | | |

For the Differentiating Infected from Vaccinated Animals (DIVA) trial the experiment was based the 23 Male entire Holstein-Friesian calves aged approximately six months of age. Nineteen cattle were vaccinated with Bacille Calmette-Guerin (BCG) after a blood sample was taken with subsequent samples taken after 2, 4 and 9 weeks. A control set of 4 cattle was sampled at similar timepoints. At 9 weeks, all cattle were infected with bovine tuberculosis and then blood was sampled at 14, 18 and 22 months. Plasma was harvested and frozen.

### Biomarker Extraction

Biomarker extraction was performed using 4:1 (v/v) methanol:chloroform. Samples were thawed on ice and vortexed, 200 μl of plasma was added to 1520 μl of the pre-chilled methanol-chloroform. Samples were then vortexed for approximately 10 seconds before being shaken for 15 minutes at 4° C. Following shaking samples were kept at −20° C. for 20 minutes before centrifugation at 18,000×g at 4° C. for 5 minutes.

100 μl of the extract was transferred to a glass vial along with 100 μl of 70:30 (v/v) methanol-water for Flow Infusion Electrospray Ionization-Mass Spectrometry (FIE-MS). Quality control (QC) samples were created for each mass spectrometry run containing equal amounts of every sample in the run diluted at a ratio 50:50 with the 70:30 (v/v) methanol-water. Samples were run on batches of varied size and where multiple batches were used, identical master-mix samples were used along with samples only with solvents as controls. Injection order of samples was randomised within and across batches, with regular injection of QC samples and control samples.

### Mass Spectrometry Data Collection and Pre-Processing

Metabolomic data was collected using a surveyor flow infusion electrospray mass spectrometry for high throughput, non-targeted metabolite fingerprinting. Data acquisition was performed for both positive and negative ionisation modes in a single run, through four different scan ranges on a Q Exactive Hybrid Quadrupole-Orbitrap Mass Spectrometer (Thermo-Scientific). Data was collected using a with positive and negative ionisations captured in a single run across four different scan ranges (110-470 m/z, 450-820 m/z, 800-1060 m/z and 1040-2000 m/z).

Lipidomic extraction was performed using n methyl tert-butyl ether (MTBE) protocol. 15 μl of plasma and 100 μl of ddH₂O were mixed in a 2 ml Eppendorf tube and centrifuged at 1500×g at 4° C. for 5 minutes. Simultaneously 500 μl of HPLC grade MTBE (Fisher Scientific), 250 μl of HPLC grade methanol and 150 μl of ddH₂O were placed in a glass vial (Fisher Scientific) and chilled on ice. 100 μl of the plasma and ddH₂O mixture was added to the glass vial, bringing the total volume to 1 ml. Samples were shaken for 10 minutes at 200×rpm at 4° C. before resting on ice until phase separation was observed. 20 μl of the organic phase (upper phase of the separation) was added to a 96 well plate (Plate+TM, Esslab, UK) containing 90 μl of 7.5 mM ammonium acetate IPA:Methanol 2:1. Sample order was randomised using the in house randomisation tool prior to plating, and samples were plated in injection order, with control (solvent only) and composite QC samples as described for the metabolomic extraction. Samples were plated and sealed immediately prior to analysis.

Median centring block correction of total ion counts was performed during the multi-batch run to account for variation in instrument performance and sensitivity over the course of runs. Relative Standard Deviation (RSD) filtering was applied using QC samples for each m/z, features with an RSD <0.5 were removed from the dataset. Occupancy filtering was performed with a ⅔ maximum occupancy requirement, consequently only a single class was required to have occupancy above the threshold for a feature to be retained. Random Forest Imputation was performed using the missForest package (Tang and Ishwaran, 2017). Data was Log ₁₀ transformed and Pareto scaled using Metaboanalyst 4.0 (Chong et al., 2020).

### Statistical Analysis

Statistical analysis was performed using R and Metaboanalyst 4.0. Principal Components Analysis (PCA) and Partial Least Squares Discriminant Analysis (PLSDA) were used to examine the overall data structure and variation both within and between experimental infections. Based upon these findings −1 and 0 weeks, along with 4 and 6-week time points were combined into pre- and post-infection classes for binary classification. Receiver operating characteristic curve (ROC) analysis was used to assess discriminatory capability of features as implemented in Metaboanalyst 4.0. ROC analysis was performed for individual markers and for combinations, or fingerprints, of promising features. ROC analysis was repeated for each targeted variable and fingerprint using experiment C as the validation test set.

### Feature Identification

The m/z ratios were used to interrogate the MZedDB database, error limits were set to 5 ppm and identification was based upon both PPM error and ionisation, prioritising protonation or deprotonation where chemically appropriate. The Metabanalyst 4.0 “Peaks to Pathways” algorithm was used alongside MZedDB searches was used to investigate variation within experiment C.

### Results

Metabolomic assessments of three separate experiments where cattle were infected with M. bovis were undertaken. Each experiment was assessed individually to indicate metabolomic changes over the course of M. bovis infection (FIG. 1).

The variables within each experiment were assessed for their ability to distinguish between pre- and post-infection using ROC analysis. When using multivariate approaches, several biomarker fingerprints of up to nine features were able to generate AUC values >0.95. The best performing panel of features (dihydro-leukotriene B4, 3-hydroxyisovaleric acid, sphingomyelin [SM(d18:2(4E,14Z)/24:0)], sphingomyelin [SM(d18:1/24:1(15Z))], 18:3 cholesteryl ester, MG(0:0/18:3(6Z,9Z,12Z)/0:0), citric acid and unknown compounds 389.21933 m/z and 886.71063 m/z (under negative ionisation)) in combination was able to generate an AUC of 0.99 in differentiating between pre-infection from post-infection samples (FIG. 2, which incorporates specificities (false discovery rates) and sensitivities (true discovery rates)). The reproducibility of the biomarkers was assessed. Samples from experiment C were extracted to be used as a validation (holdout) set. Using this approach, experiments A and B combined (CV) had and AUC value of >0.99 and experiment C (Holdout) a value of 0.98. This indicated a high degree of reproducibility for the biomarker fingerprint across the experiments (FIG. 3). The identities of the features and their ionisation characteristics are summarised in Table 2.

TABLE 2
Fingerprint feature characteristics and identifications

| | | | | Log2 Fold | |
|---|---|---|---|---|---|
| m/z | Ionisation | AUC | p-value | Change | ID |
| 117.05547 | Negative | 0.87349 | 3.59E−18 | −0.87423 | 3-Hydroxyisovaleric acid |
| 337.23959 | Negative | 0.86967 | 5.03E−17 | 0.92932 | Dihydro-leukotrieneB4 |
| 863.67865 | Negative | 0.84599 | 1.92E−09 | 0.59867 | sphingomyelin |
| | | | | | [SM(d18:2(4E,14Z)/24:0)], |
| 389.21933 | Negative | 0.83908 | 2.34E−16 | −0.52518 | unknown |
| 811.67065 | Negative | 0.81638 | 2.92E−09 | 0.77976 | Sphingomyelin |
| | | | | | [SM(d18:1/24:1(15Z))] |
| 664.60254 | Positive | 0.81303 | 1.67E−10 | 0.5621 | 18:3 Cholesteryl ester |
| 886.71063 | Negative | 0.83711 | 1.6E−13 | 0.62477 | unknown |
| 351.2554 | Negative | 0.77724 | 5.5722E−6 | 0.97071 | MG(0:0/18:3(6Z,9Z,12Z)/0:0) |
| 191.01945 | Negative | 0.84441 | 7.74E−13 | −0.9905 | Citric acid |

For the DIVA experiment, several metabolites did not show changes in the BOG vaccinated cattle group but did show changes following challenge with M. bovis (Table 3). Dihydro-leukotriene B4, pyrocatechol, MG(0:0/18:3(6Z,9Z,12Z)/0:0), N-(docosanoyl)-heptadecasphing-4-enine-1-phosphocholine, 3-hydroxyisovaleric acid, sphingomyelin [SM(d18:2(4E,14Z)/24:0)], 18:3 cholesteryl ester, citric acid and unknown compounds 359.22107 m/z and 389.21933 m/z (under negative ionisation). were the best performing.

TABLE 3
Metabolites with high discriminatory capability between
vaccinated and Vaccinated + Infected samples

| | | | | Log2 Fold | |
|---|---|---|---|---|---|
| m/z | Ionisation | AUC | p-value | Change | ID |
| 337.23978 | Negative | 0.9473 | 8.30E−09 | 6.363 | Dihydro-leukotrieneB4 |
| 109.02934 | Negative | 0.90859 | 2.77E−05 | −99 | Pyrocatechol |
| 351.25519 | Negative | 0.90582 | 1.00E−06 | −99 | MG(0:0/18:3(6Z,9Z,12Z)/0:0) |
| 795.63641 | Positive | 0.94598 | 4.59E−06 | −99 | N-(docosanoyl)- |
| | | | | | heptadecasphing-4-enine-1- |
| | | | | | phosphocholine |
| 359.22107 | Negative | 0.93352 | 8.48E−07 | 2.678 | Unknown |
| 389.21933 | Negative | 0.83656 | 2.34E−16 | −0.85639 | Unknown |
| 117.05553 | Negative | 0.7257 | 2.92E−09 | 0.2411 | 3-Hydroxyisovaleric acid |
| 863.67865 | Negative | 0.7 | 1.67E−10 | −0.23349 | sphingomyelin |
| | | | | | [SM(d18:2(4E,14Z)/24:0)], |
| 664.60254 | Positive | 0.69259 | 1.6E−13 | 0.25107 | 18:3 Cholesteryl ester |
| 117.05553 | Negative | 0.72576 | 5.5722E−6 | 0.2411 | Citric acid |

### DISCUSSION

In this study the inventors have demonstrated that particular biomarkers can be used to differentiate between healthy and M. bovis infected cattle. The inventors have shown that biomarkers dihydro-leukotriene B4, 3-hydroxyisovaleric acid, sphingomyelin [SM(d18:2(4E,14Z)/24:0)], sphingomyelin [SM(d18:1/24:1(15Z))], 18:3 cholesteryl ester, MG(0:0/18:3(6Z,9Z,12Z)/0:0), citric acid and unknown compounds 389.21933 m/z and 886.71063 m/z (under negative ionisation), are particularly effective in differentiating between healthy and M. bovis infected cattle. By utilising combinations of these biomarkers, highly discriminatory and reproducible results were obtained in three separate groups of animals, indicating the diagnostic potential of these biomarkers. The predominant current diagnostic techniques are based upon stimulation of cell-mediated immune responses but the inventors show here the capability to identify markers in plasma without in vivo or in vitro stimulation. Therefore, a diagnostic test based on the biomarkers disclosed herein has the potential to simplify the diagnostic process. Such a test system would result in a much-improved accuracy compared to current diagnostic testing approaches.

The use of three independent groups, totalling 61 animals, has allowed the inventors to identify the robust and repeatable biomarker variables attributable to M. bovis infection.

For the Differentiating Infected from Vaccinated Animals (DIVA) trial, the following biomarkers were identified as changing only on infection and not during vaccination (Table 3): dihydro-leukotriene B4, pyrocatechol, MG(0:0/18:3(6Z,9Z,12Z)/0:0), N-(docosanoyl)-heptadecasphing-4-enine-1-phosphocholine, 3-hydroxyisovaleric acid, sphingomyelin [SM(d18:2(4E,14Z)/24:0)], 18:3 cholesteryl ester, citric acid and unknown compounds 359.22107 m/z and 389.21933 m/z (under negative ionisation). These biomarkers can therefore be used to accurately distinguish between animals which have been vaccinated against M. bovis and those which are also infected.

The specific combination of dihydro-leukotriene B4, MG(0:0/18:3(6Z,9Z,12Z)/0:0), 3-hydroxyisovaleric acid, sphingomyelin [SM(d18:2(4E,14Z)/24:0)], 18:3 cholesteryl ester, citric acid and unknown compound 389.21933 m/z (under negative ionisation) have combined AUCs in excess of 0.99 for both discriminating infected and uninfected cattle and differentiating infected from vaccinated samples. Crucially, this seven-feature panel can identify infection in vaccinated animals, where protection appears to have failed.

### CONCLUSION

Analysis of heifers infected with M. bovis and control cattle, identified several biomarkers which show clear differentiation between M. bovis infected and control cattle, including biomarkers dihydro-leukotriene B4, 3-hydroxyisovaleric acid, sphingomyelin [SM(d18:2(4E,14Z)/24:0)], sphingomyelin [SM(d18:1/24:1(15Z))], 18:3 cholesteryl ester, MG(0:0/18:3(6Z,9Z,12Z)/0:0), citric acid and two unknown compounds 389.21933 m/z and 886.71063 m/z in negative ionisation which were present at significantly different levels in the M. bovis infected cattle.

Taken together, combinations of the nine identified metabolites, dihydro-leukotriene B4, 3-hydroxyisovaleric acid, sphingomyelin [SM(d18:2(4E,14Z)/24:0)], sphingomyelin [SM(d18:1/24:1(15Z))], 18:3 cholesteryl ester, MG(0:0/18:3(6Z,9Z,12Z)/0:0), citric acid and unknown compounds 389.21933 m/z and 886.71063 m/z (under negative ionisation) represent a collection of biomarker signatures which can target M. bovis infected cattle with a pooled accuracy of >99%. These metabolites demonstrate greater combined sensitivity and specificity when predicting M. bovis infection than the commercial diagnostic tests currently available and may therefore be extremely effective biomarkers for use in identifying M. bovis infections.

The inventors also identified biomarkers dihydro-leukotriene B4, pyrocatechol, MG(0:0/18:3(6Z,9Z,12Z)/0:0), N-(docosanoyl)-heptadecasphing-4-enine-1-phosphocholine, 3-hydroxyisovaleric acid, sphingomyelin [SM(d18:2(4E,14Z)/24:0)], 18:3 cholesteryl ester, citric acid and unknown compounds 359.22107 m/z and 389.21933 m/z (under negative ionisation) which were present at significantly different levels in cattle infected with M. bovis as opposed to cattle vaccinated against M. bovis. The ability to differentiate cattle infected with bovine tuberculosis from vaccinated cattle is particularly advantageous.

It will be appreciated that numerous modifications to the above-described method, including smaller and alternative selections of the identified biomarkers, may be made without departing from the scope of the invention as defined in the appended claims.

### REFERENCES

Chong, J., Wishart, D. S., & Xia, J. (2019). Using metaboanalyst 4.0 for comprehensive and integrative metabolomics data analysis. Current Protocols in Bioinformatics, 68, e86. doi: 10.1002/cpbi.86
Tang, F, Ishwaran, H. Random Forest Missing Data Algorithms. Stat Anal Data Min: The ASA Data Sci Journal. 2017; 10: 363-377. https://doi.org/10.1002/sam.11348.

1. A method for determining the presence of Mycobacterium bovis in a subject, the method comprising the steps of:
(i) determining the level of one or more biomarkers in a sample from the subject;
(ii) comparing the level of said one or more biomarkers with the level of said one or more biomarkers in a control sample to determine whether Mycobacterium bovis is present in the subject;
wherein the one or more biomarkers are selected from: dihydro-leukotriene B4, 3-hydroxyisovaleric acid, sphingomyelin [SM(d18:2(4E,14Z)/24:0)], sphingomyelin [SM(d18:1/24:1(15Z))], 18:3 cholesteryl ester, an unknown compound 389.21933 m/z (under negative ionisation), an unknown compound 886.71063 m/z (under negative ionisation), citric acid, MG(0:0/18:3(6Z,9Z,12Z)/0:0), pyrocatechol, N-(docosanoyl)-heptadecasphing-4-enine-1-phosphocholine and an unknown compound 359.22107 m/z (under negative ionisation).

2. The method according to claim 1, wherein the one or more biomarkers are selected from: dihydro-leukotriene B4, 3-hydroxyisovaleric acid, sphingomyelin [SM(d18:2(4E,14Z)/24:0)], sphingomyelin [SM(d18:1/24:1(15Z))], 18:3 cholesteryl ester, an unknown compound 389.21933 m z (under negative ionisation), an unknown compound 886.71063 m/z (under negative ionisation), citric acid and MG(0:0/18:3(6Z,9Z,12Z)/0:0.

3. The method according to claim 1, wherein when the one or more biomarkers are selected from 3-hydroxyisovaleric acid, unknown compound 389.21933 m/z (under negative ionisation) and citric acid a biomarker level in the sample from the subject which is lower than the biomarker level in the control sample indicates that Mycobacterium bovis is present in the subject.

4. The method according to claim 1, wherein when the one or more biomarkers comprises dihydro-leukotriene B4, sphingomyelin [SM(d18:2(4E,14Z)/24:0)], sphingomyelin [SM(d18:1/24:1(15Z))],18:3 cholesteryl ester, unknown compound 866.71063 m/z (under negative ionisation) or MG(0:0/18:3(6Z,9Z,12Z)/0:0). a biomarker level in the sample from the subject which is higher than the biomarker level in the control sample indicates that Mycobacterium bovis is present in the subject.

5. The method according to claim 1, wherein the method is for distinguishing between subjects both infected with and vaccinated against Mycobacterium bovis and subjects vaccinated against Mycobacterium bovis.

6. The method according to claim 5, wherein the one or more biomarkers are selected from dihydro-leukotriene B4, pyrocatechol, MG(0:0/18:3(6Z,9Z,12Z)/0:0), N-(docosanoyl)-heptadecasphing-4-enine-1-phosphocholine, 3-hydroxyisovaleric acid, sphingomyelin [SM(d18:2(4E,14Z)/24:0)], 18:3 cholesteryl ester, citric acid, an unknown compound 389.21933 m/z (under negative ionisation) and an unknown compound 359.22107 m/z (under negative ionisation).

7. The method according to claim 6 wherein when the one or more biomarkers are selected from dihydro-leukotriene B4, an unknown compound 359.22107 m/z (under negative ionisation), 3-hydroxyisovaleric acid, citric acid and 18:3 cholesteryl ester, a biomarker level in the sample which is higher than the biomarker level in the control sample indicates that the subject is infected with Mycobacterium bovis; and/or
wherein when the one or more biomarkers are selected from pyrocatechol, MG(0:0/18:3(6Z,9Z,12Z)/0:0), N-(docosanoyl)-heptadecasphing-4-enine-1-phosphocholine, unknown compound 389.21933 m/z (under negative ionisation), sphingomyelin [SM(d18:2(4E,14Z)/24:0)], a biomarker level in the sample which is lower than the biomarker level in the control sample indicates that the subject is infected with Mycobacterium bovis.

8. The method according to claim 1, wherein the biomarkers consist of dihydro-leukotriene B4, MG(0:0/18:3(6Z,9Z,12Z)/0:0), 3-hydroxyisovaleric acid, sphingomyelin [SM(d18:2(4E,14Z)/24:0)], 18:3 cholesteryl ester, citric acid and unknown feature 886.71063 m/z (under negative ionisation).

9. The method according to claim 1, wherein the subject is cattle.

10. The method according to claim 1, wherein the method further comprises providing a sample from the subject.

11. The method according to claim 1, wherein the method comprises a method for determining the presence of bovine tuberculosis in a subject.

12. An immunological capture device for detecting Mycobacterium bovis in a subject, the device comprising a substrate carrying capture antibodies to one or more of the following biomarkers: dihydro-leukotriene B4, 3-hydroxyisovaleric acid, sphingomyelin [SM(d18:2(4E,14Z)/24:0)], sphingomyelin [SM(d18:1/24:1(15Z))], 18:3 cholesteryl ester, an unknown compound 389.21933 m z (under negative ionisation), an unknown compound 886.71063 m/z (under negative ionisation), citric acid, MG(0:0/18:3(6Z,9Z,12Z)/0:0), pyrocatechol, N-(docosanoyl)-heptadecasphing-4-enine-1-phosphocholine and an unknown compound 359.22107 m/z (under negative ionisation).

13. The immunological capture device according to claim 12, wherein the device is for detecting bovine tuberculosis in a subject.

14. The immunological capture device according to claim 13, wherein the substrate carries capture antibodies to one or more of the following biomarkers: dihydro-leukotriene B4, 3-hydroxyisovaleric acid, sphingomyelin [SM(d18:2(4E,14Z)/24:0)], sphingomyelin [SM(d18:1/24:1(15Z))], 18:3 cholesteryl ester, an unknown compound 389.21933 m/z (under negative ionisation), an unknown compound 886.71063 m/z (under negative ionisation), citric acid and MG(0:0/18:3(6Z,9Z,12Z)/0:0.

15. The immunological capture device according to claim 13, wherein the substrate carries capture antibodies to dihydro-leukotriene B4, 3-hydroxyisovaleric acid, sphingomyelin [SM(d18:2(4E,14Z)/24:0)], sphingomyelin [SM(d18:1/24:1(15Z))], 18:3 cholesteryl ester, an unknown compound 389.21933 m/z (under negative ionisation), an unknown compound 886.71063 m/z (under negative ionisation), citric acid and MG(0:0/18:3(6Z,9Z,12Z)/0:0.

16. The immunological capture device according to claim 12, wherein the device is for distinguishing between subjects both infected with and vaccinated against Mycobacterium bovis and subjects vaccinated against Mycobacterium bovis.

17. The immunological capture device according to claim 16, wherein the substrate carries capture antibodies to one or more of the following biomarkers: dihydro-leukotriene B4, pyrocatechol, MG(0:0/18:3(6Z,9Z,12Z)/0:0), N-(docosanoyl)-heptadecasphing-4-enine-1-phosphocholine, 3-hydroxyisovaleric acid, sphingomyelin [SM(d18:2(4E,14Z)/24:0)], 18:3 cholesteryl ester, citric acid, an unknown compound 389.21933 m/z (under negative ionisation) and an unknown compound 359.22107 m/z (under negative ionisation).

18. The immunological capture device according to claim 12, wherein the substrate carries capture antibodies to the following biomarkers: dihydro-leukotriene B4, MG(0:0/18:3(6Z,9Z,12Z)/0:0), 3-hydroxyisovaleric acid, sphingomyelin [SM(d18:2(4E,14Z)/24:0)], 18:3 cholesteryl ester, citric acid and unknown compound 886.71063 m/z (under negative ionisation).

19. A kit for determining the presence of Mycobacterium bovis in a subject, the kit comprising:
(i) an immunological capture device comprising a substrate carrying capture antibodies to one or more of the following biomarkers: dihydro-leukotriene B4, 3-hydroxyisovaleric acid, sphingomyelin [SM(d18:2(4E,14Z)/24:0)], sphingomyelin [SM(d18:1/24:1(15Z))], 18:3 cholesteryl ester, an unknown compound 389.21933 m/z (under negative ionisation), an unknown compound 886.71063 m/z (under negative ionisation), citric acid, MG(0:0/18:3(6Z,9Z,12Z)/0:0), pyrocatechol, N-(docosanoyl)-heptadecasphing-4-enine-1-phosphocholine and an unknown compound 359.22107 m/z (under negative ionisation); and
(ii) a second antibody, wherein the second antibody is specific to the one or more biomarkers or to the capture antibodies.

20. The kit according to claim 19 wherein the second antibody comprises a coloured particle covalently linked to the second antibody.
